(19) United States
(12) Patent Application Publication
(10) Pub. No.: US 2017/0196735 A1
(43) Pub. Date: Jul. 13, 2017

(54) CLOSED-WOUND DRAINAGE DEVICE AND OPERATION METHOD

(71) Applicant: FORTUNE MEDICAL INSTRUMENT CORP., New Taipei City (TW)

(72) Inventors: Shun-Hsien WANG, New Taipei City (TW); Tai-Li WANG, New Taipei City (TW)

(21) Appl. No.: 15/403,049

(22) Filed: Jan. 10, 2017

(30) Foreign Application Priority Data
Jan. 12, 2016 (TW) 105100883

(51) Int. Cl. A61F 13/00; A61M 1/00; A61F 13/02

(52) U.S. Cl. CPC A61F 13/00068; A61F 13/00063; A61F 13/0216; A61M 1/0088; A61M 1/0084; A61F 2013/0028

(57) ABSTRACT

A closed-wound drainage device is provided in the following two forms: (1) an irregularly-shaped foam drug-containing coverage material, a drainage tube, a canister and a pressure source and (2) a flat foam drug-containing coverage material, a drainage tube, a canister and a pressure source. The function of which is to cover the wound and to drain, promote cell growth and accelerate wound healing when the pressure source operates. Its operation method is as follows: put the irregularly-shaped or flat foam drug-containing coverage material on the wound so that the adhesive layer is attached to normal skin, then connect the drainage tube with the canister and the pressure source. When the pressure source activates, the irregularly-shaped or flat foam drug-containing coverage material is attached to the surface of the wound to accelerate recovery of the wound.

This application claims priority to Taiwan Patent Application No. 105100883 filed on Jan. 12, 2016, which is hereby incorporated by reference in its entirety.

### CROSS-REFERENCES TO RELATED APPLICATIONS

Not applicable.

### BACKGROUND OF THE INVENTION

Field of the Invention

The present invention provides a closed-wound drainage device that can promote the growth of tissue to accelerate the recovery of a wound.

Descriptions of the Related Art

Currently, in clinical practice, a foam dressing is used to apply a reduced pressure at the site of a wound. The foam dressing comes into contact with the wound to distribute the reduced pressure, with a size suitable for the current wound. As the wound begins to heal and becomes smaller, the foam dressing needs to be periodically replaced with smaller foam dressings. However, replacing the foam dressing frequently causes pain and discomfort to the user.

Decompression treatment usually applies to non-healing open wounds. These tissues can be subcutaneous, or within or on the skin tissue. Conventionally, decompression treatment has been mainly applied on soft tissues, and has not been applied to treat closed wounds deep in the tissues of diabetic patients because it is difficult to form a foam dressing that fits the shapes of such wounds. In addition, the foam dressing must be handmade into a shape suitable for the wound site and then removed after a decompression treatment period. Because the wound is covered by the foam dressing in the process of the decompression treatment period, it is difficult to observe the conditions of the granulation tissue growth of the wound.

### SUMMARY OF THE INVENTION

To solve the problem with conventional wound treating systems and methods, a closed-wound drainage device for applying a reduced pressure to a tissue site is provided in the present invention. The closed-wound drainage device comprises an irregularly-shaped or flat foam drug-containing coverage material, a drainage tube, an adhesive layer, a canister and a pressure source.

In the first implementation, the irregularly-shaped or flat foam drug-containing coverage material comprises a flat coverage material, a foam, and an adhesive layer. The foam and the adhesive layer are formed directly on the flat coverage material. The flat coverage material has an elastic property, which can be formed into the shape and size of the wound tissue site when being pumped by the pressure source. The irregularly-shaped or flat foam drug-containing coverage material of this implementation is only suitable for shallow wound tissues. The material is biocompatible, so it has no adverse effect on the wound tissue. Foam gaps in the foam can function as drainage channels, as well as drug carriers to drain liquid wastes and to diffuse the drug so that better drainage and drug treatment effects can be obtained to promote cell growth and an antibacterial effect.

In the second implementation, the irregularly-shaped or flat foam drug-containing coverage material comprises an irregularly-shaped coverage material, a foam, and an adhesive layer. The irregularly-shaped coverage material comprises one or more irregularly-shaped flexible loops that are adaptable to different shapes, sizes, and depths of the wound tissue. The material is biocompatible, so it has no adverse effect on the wound tissue. Foam gaps in the foam can function as drainage channels, as well as drug carriers to drain liquid wastes and to diffuse the drug so that better drainage and drug treatment effects can be obtained to promote cell growth and an antibacterial effect.

An adhesive layer is disposed in the outermost groove of the irregularly-shaped or flat foam drug-containing coverage material. The irregularly-shaped or flat foam drug-containing coverage material is attached on the normal skin of the user by the adhesive layer. The adhesion can be airtight when it is pumped by the pressure source to effectively create a negative-pressure environment. The adhesive layer itself is biocompatible, so it will not cause irritation to the normal skin. Furthermore, the adhesive layer helps keep the wound tissue in a negative-pressure environment.

A drainage tube comprises a drainage port and an injection and washing port. The drainage port comprises a plurality of heteromorphic protrusions. Under the action of the pressure source or when being pressed by an external force from outside the wound tissue, the heteromorphic protrusions in the drainage port will support the tube wall of the drainage tube to form a triangle space (gap) between the tube wall and the heteromorphic protrusions so that space for drainage may still remain when the drainage tube is pressed. When it is necessary to wash the necrotic cells that are generated during the treatment process or to inject a drug to enhance the treatment, the injection and washing port of the drainage tube can be used to perform the washing operation or drug injection operation.

A canister collects the wasted tissue liquid and hardens the wasted tissue liquid by using a chemical agent to prevent environmental pollution and damage of the pressure source.

A pressure source functions as producing a negative-pressure effect that allows the wound tissue to stay in a negative-pressure treatment environment, and provides a continuous or intermittent pressure to promote growth of the wound tissue.

A closed-wound drainage operation method comprises the following steps: tearing off a protective film from a closed-wound drainage device; attaching the closed-wound drainage device on a wound tissue and attaching an adhesive layer of a drug-containing coverage material on a normal skin to keep air tightness therebetween; connecting a drainage tube to a canister; connecting the canister to a pressure source; activating the pressure source to form a negative-pressure treatment environment in the wound tissue by the closed-wound drainage device; and then observing the recovery conditions of the wound through the window of the drug-containing coverage material, and if necessary, performing a washing or drug injection operation via the injection and washing port to accelerate the recovery of the wound.

### BRIEF DESCRIPTION OF THE DRAWINGS

FIG. 1 is a schematic view of a conventional negative-pressure treatment, drainage and pumping device;

FIG. 2 is a schematic exploded view of an irregularly-shaped or flat closed-wound drainage device;

FIG. 3 is a schematic view of an embodiment of the closed-wound drainage device;

FIG. 4 is an exploded view of an irregularly-shaped foamed coverage material and a flat foamed coverage material;

FIG. 5 is a cross-sectional view of the irregularly-shaped foamed coverage material;

FIG. 6 is a cross-sectional view of the flat foamed coverage material;

FIG. 7 is an enlarged schematic view of the irregularly-shaped or flat foamed coverage material according to an embodiment;

FIG. 8 is a schematic view illustrating the positions of the irregularly-shaped coverage material and the foam according to an embodiment;

FIG. 9 is a schematic view illustrating the positions of the flat coverage material and the foam according to an embodiment;

FIG. 10 is a schematic view illustrating a case where the foam is distributed throughout the coverage material according to an embodiment;

FIG. 11 is a schematic view illustrating a case where the foam is uniformly distributed in the coverage material according to an embodiment;

FIG. 12 is a schematic view illustrating a case where the foam is non-uniformly distributed in the coverage material according to an embodiment;

FIG. 13 is a schematic view illustrating dead spaces in the irregularly-shaped or flat coverage material;

FIG. 14 is a schematic view illustrating the shape-forming of the irregularly-shaped or flat coverage material;

FIG. 15 is a schematic view of a drainage tube;

FIG. 16 is a schematic view of the irregularly-shaped coverage material of the closed-wound drainage device according to an embodiment;

FIG. 17 is a schematic cross-sectional view of FIG. 16 taken along a cross-sectional line 17-17;

FIG. 18 is a schematic view of a flat coverage material of the closed-wound drainage device according to an embodiment;

FIG. 19 is a schematic cross-sectional view of FIG. 18 taken along a cross-sectional line 19-19; and

FIG. 20 is a schematic view of a flowchart diagram of a closed-wound drainage operation method.

### DESCRIPTION OF THE PREFERRED EMBODIMENT

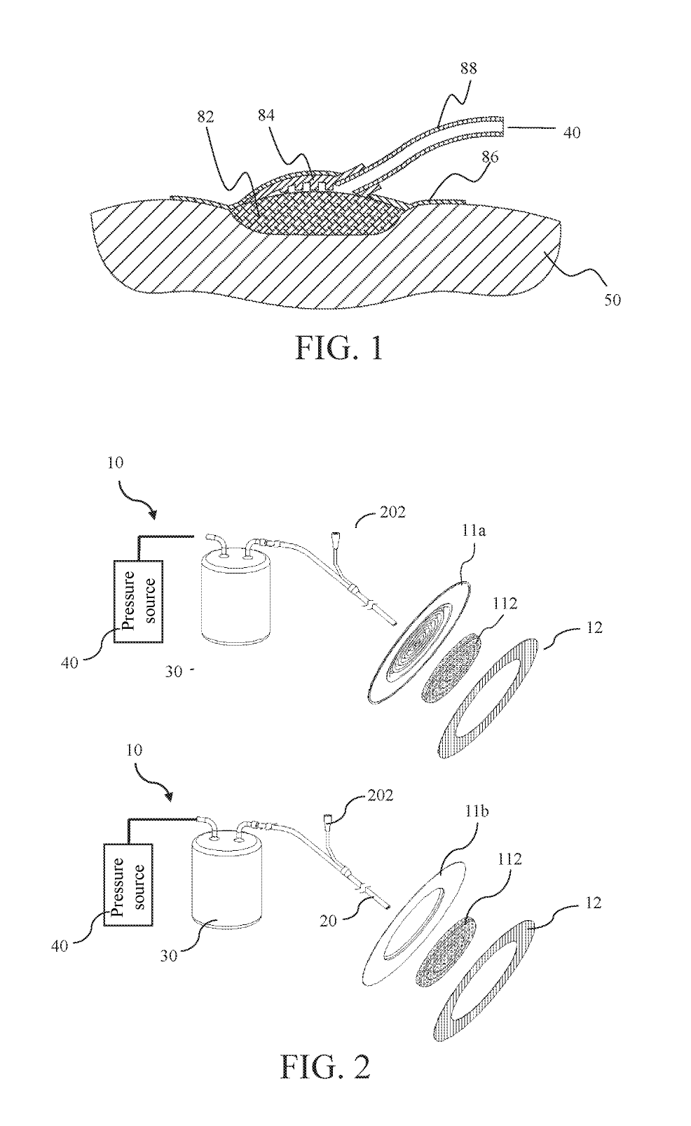

A general conventional negative-pressure wound treatment, drainage and pumping device is as shown in FIG. 1. Primarily, in the current clinical practices of negative-pressure wound treatment, a foam dressing 82, a flexible sucking pad 84 and a transparent adhesive film 86 are put on a wound and a pressure source 40 is connected to create a negative-pressure environment 60 in the wound that helps to pump out the infectious substances 70 to accelerate the recovery of the wound.

FIG. 2 is a schematic exploded view of an irregularly-shape or flat closed-wound drainage device, while FIG. 3 is a schematic view of an embodiment of the closed-wound drainage device. The closed-wound drainage device 10 comprises the following components: an irregularly-shaped or flat foam drug-containing coverage material 11, a drainage tube 20, a canister 30, and a pressure source 40 connected together. When the closed-wound drainage device 10 is attached on a wound tissue 50, an adhesive layer 12 adheres to normal skin 80 to provide an airtight effect. The canister 30 is connected with the pressure source 40 via the drainage tube 20 to create a complete negative-pressure environment 60 for treating the wound tissue 50. The infectious substances 70 generated in the wound tissue 50 can be pumped into the canister 30 to promote blood microcirculations around the wound tissue 50 and, thus, accelerate the recovery of the wound.

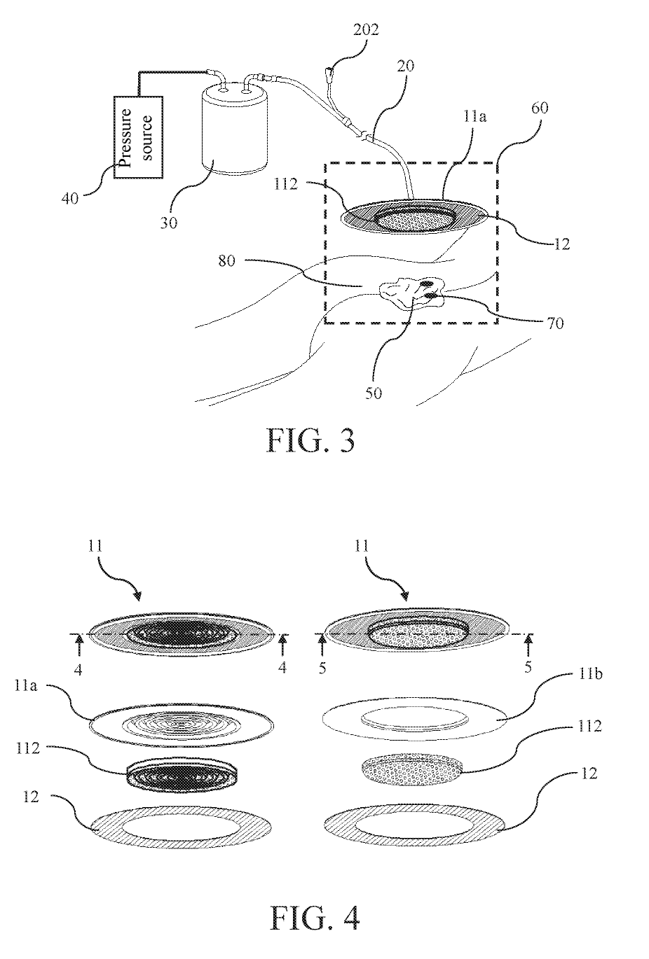

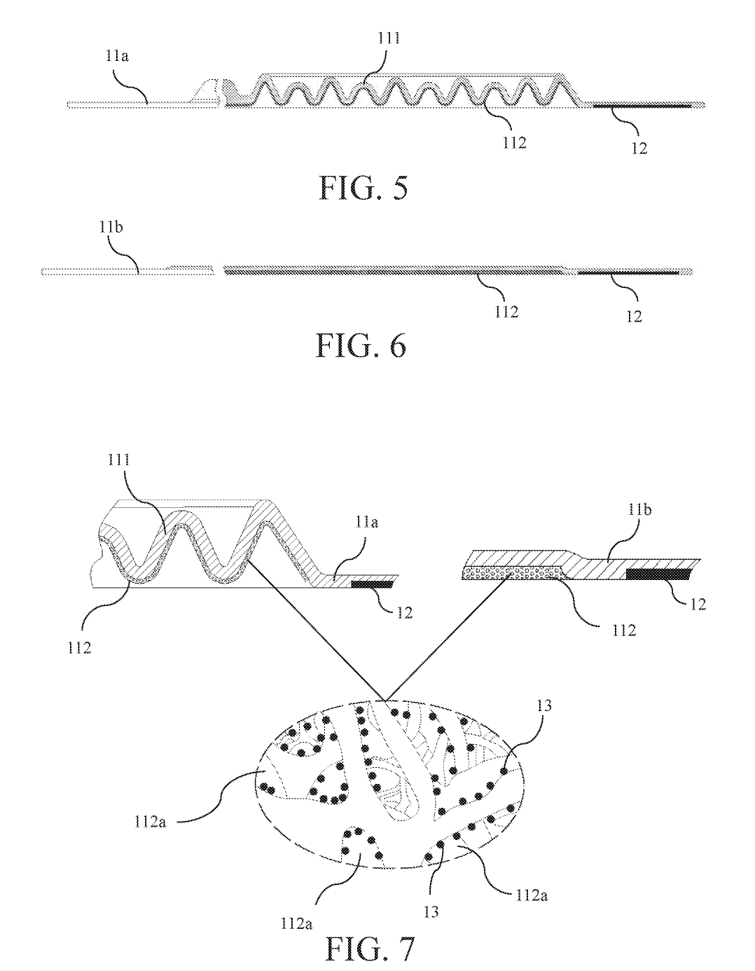

FIG. 4 is an exploded view of an irregularly-shaped foamed coverage material and a flat foamed coverage material, FIG. 5 is a cross-sectional view of the irregularly-shaped foamed coverage material, and FIG. 6 is a cross-sectional view of the flat foamed coverage material. In the first implementation, an irregularly-shaped or flat foam drug-containing coverage material 11 comprises an irregularly-shaped coverage material 11a, a foam 112, and an adhesive layer 12. Firstly, the irregularly-shaped coverage material 11a comprises one or more irregularly-shaped flexible loops 111 adaptable to deeper wound tissues. In this embodiment, silicone is used as the material of the irregularly-shaped coverage material 11a to describe the function and property of the irregularly-shaped coverage material 11a. When the irregularly-shaped coverage material 11a has a hardness ranging between Shore A 20 and Shore A 80 and a thickness ranging between 0.05 mm and 2 mm, the irregularly-shaped coverage material 11a is suitable for wounds with a size ranging between 6 cm² and 400 cm². Through pumping by the pressure source 40 and extension of the irregularly-shaped flexible loops 111, the irregularly-shaped coverage material 11a can be attached on the wound tissue 50 with a deeper depth regardless of the size of the wound tissue. Secondly, the foam 112 formed integrally with the irregularly-shaped coverage material 11a may function as drainage channels as well as drug carriers and can be attached on the wound along with the irregularly-shaped coverage material 11a. Thirdly, an adhesive layer is attached on the skin of the user to reach an airtight condition.

In the second implementation, the irregularly-shaped or flat foam drug-containing coverage material 11 comprises a flat coverage material 11b, a foam 112, and an adhesive layer 12. First, the flat coverage material 11b is designed to be flat, which is adaptable to shallower wound tissues. In this embodiment, silicone is taken as an example of the material of the irregularly-shaped coverage material 11b to describe the function and property of the irregularly-shaped coverage material 11b. When the irregularly-shaped coverage material 11b has a hardness ranging between Shore A 20 and Shore A 80 and a thickness ranging between 0.05 mm and 2 mm, the irregularly-shaped coverage material 11b is suitable for wounds with a size ranging between 6 cm² and 400 cm² and a wound depth between 1 mm and 30 mm. Through pumping by the pressure source 40, the irregularly-shaped coverage material 11a can spread to different sites of the wound tissue 50 by the elasticity of silicone. Secondly, the foam 112 formed integrally with the irregularly-shaped coverage material 11b may function as drainage channels, as well as a drug carrier and can be attached on the wound along with the irregularly-shaped coverage material 11b. Thirdly, an adhesive layer is attached on the skin of the user to reach an airtight condition.

Please refer to FIG. 7, which is an enlarged schematic view of the irregularly-shaped or flat foam coverage material according to an embodiment. The foam 112 of the irregularly-shaped or flat foam drug-containing coverage material 11 may function as drainage channels as well as a drug carrier. Through the pumping by the pressure source 40, the foam 112 is attached on the wound tissue 50 along with the extension of the irregularly-shaped or flat foam drug-containing coverage material 11. First, in place of the conventional foam or gauze dressing and patches formed with protrusions and draining grooves, the foam gaps 112a are used to drain wasted liquid to accelerate recovery of the wound tissue. Secondly, the foam 112 may also function as a drug carrier by containing the drug 13 into the foam gaps 112 or molecular structures of the foam 112 in advance. The drug 13 has cell growth promotion or antibacterial effects, and when pumped by the pressure source 40, the drug contained in the foam 112 diffuses to different sites of the wound tissue 50 to provide cell growth promotion and antibacterial effects.

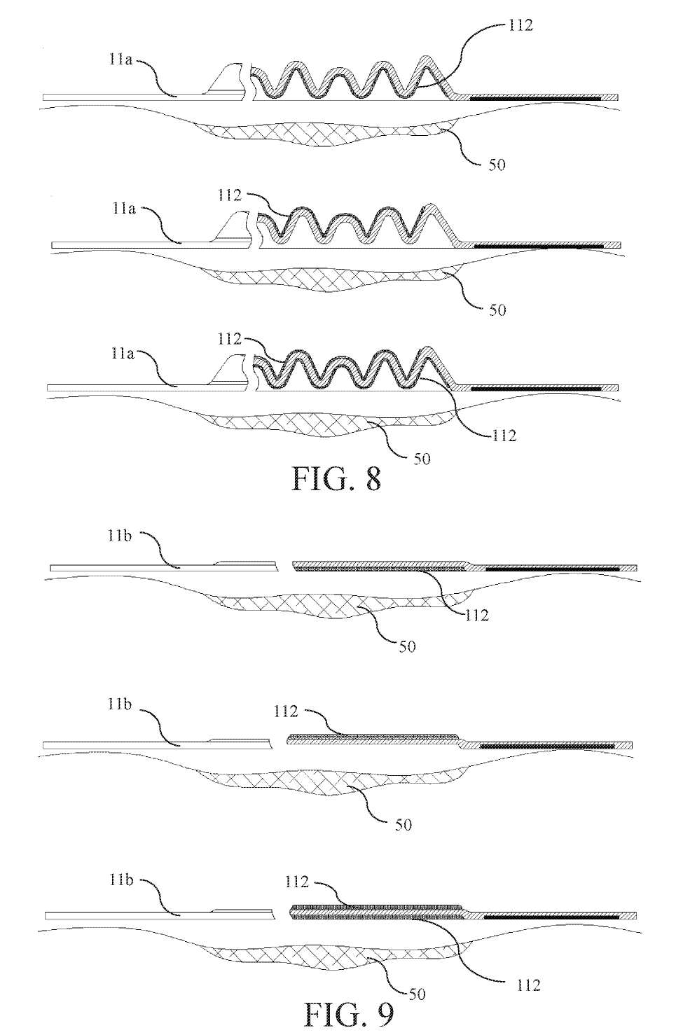

Please refer to FIG. 8, which is a schematic view illustrating the positions of the irregularly-shaped coverage material and the foam according to an embodiment. The irregularly-shaped coverage material 11a and the foam 112 may be foamed at the following locations: (1) a surface where the irregularly-shaped coverage material 11a comes into contact with the wound tissue 50, (2) a surface where the irregularly-shaped coverage material 11a makes no contact with the wound tissue 50, and (3) both the surfaces where the irregularly-shaped coverage material 11a makes or does not make contact with the wound tissue 50. The foaming area may range between 6 cm² and 400 cm² and the foaming depth may range between 0.5 mm and 20 mm.

Please refer to FIG. 9, which is a schematic view illustrating positions of the flat coverage material and the foam according to an embodiment. The flat coverage material 11b and the foam 112 may be foamed at the following locations: (1) a surface where the flat coverage material 11b makes contact with the wound tissue 50, (2) a surface where the flat coverage material 11b makes no contact with the wound tissue 50, and (3) both the surfaces where the flat coverage material 11b makes or does not make contact with the wound tissue 50. The foaming area may range between 6 cm² and 400 cm² and the foaming depth may range between 0.5 mm and 20 mm.

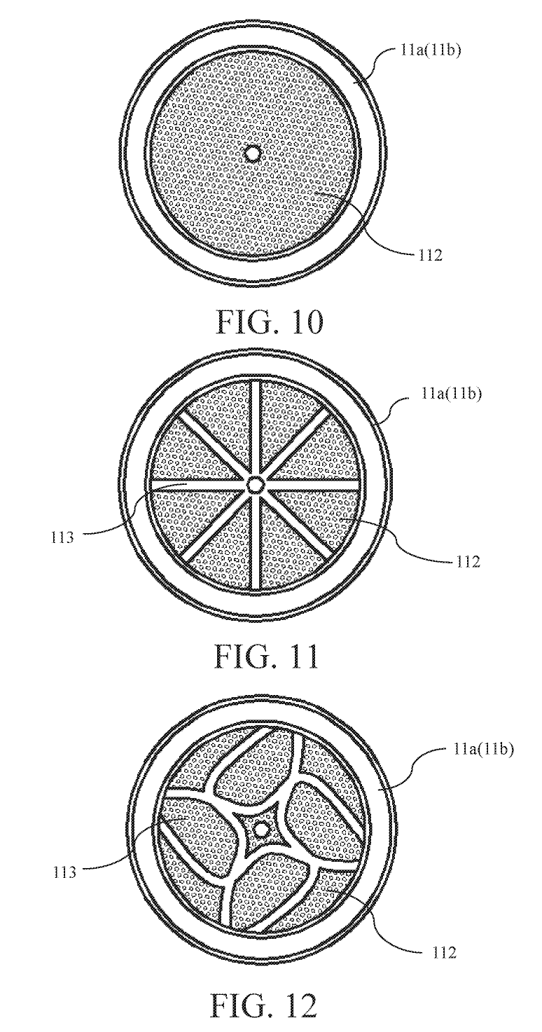

Please refer to FIG. 10, FIG. 11 and FIG. 12. FIG. 10 is a schematic view illustrating a case where the foam is distributed throughout the coverage material according to an embodiment. FIG. 11 is a schematic view illustrating a case where the foam is uniformly distributed in the coverage material according to an embodiment. FIG. 12 is a schematic view illustrating a case where the foam is non-uniformly distributed in the coverage material according to an embodiment. The foam 112 may be foamed on the irregularly-shaped coverage material 11a or the flat coverage material 11b in the following foam distribution forms: an overall distribution, a uniform distribution and a non-uniform distribution. The purpose of uniform distribution and non-uniform distribution is that, due to the transparency of the silicone itself, portions where the foam 112 is not foamed can function as a window 113 for observing the recovery conditions of the wound. The window 113 has a width ranging between 2 mm and 15 mm. The window 113 may also be formed in other ways instead of by the foam 112, e.g., the window 113 may be formed by a chemically etched (etched texture) surface or through sandblasting.

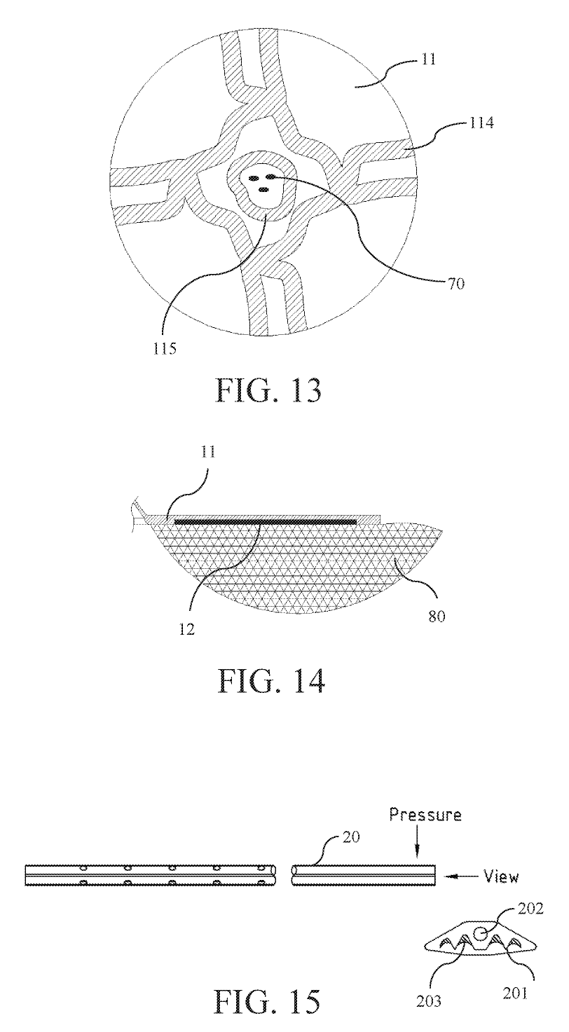

FIG. 13 is a schematic view illustrating dead spaces in the irregularly-shaped or flat coverage material. The irregularly-shaped coverage material 11 or the flat coverage material 11′ is formed to have a chemically etched (etched texture) surface free of dead spaces, where the etched traces 114 free of dead spaces shall communicate with each other to avoid formation of any dead space 115 due to isolated etched traces. The etched traces 114 free of dead spaces may function as negative-pressure drainage channels to avoid retention of infectious substances of the wound tissue in the dead spaces 115. The etched texture has a texture width ranging between 0.01 mm and 5 mm and a depth ranging between 0.01 mm and 5 mm.

FIG. 14 is a schematic view illustrating the shape-forming of the irregularly-shaped or flat coverage material. The irregularly-shaped or flat foam drug-containing coverage material 11 is attached on the skin tissue 80 by means of the adhesive layer 12 to provide a completely airtight condition. The adhesive layer 12 is made of a medical-grade silicone which is biocompatible, so it will not have any adverse effect on the normal tissue and, as proven by clinical experiments, will not cause irritation or inflammation to the normal tissue.

FIG. 15 is a schematic view of a drainage tube. The drainage tube 20 of the present invention has a plurality of heteromorphic protrusions 201 which functions to support the tube wall to form a triangle space (gap) 203 in the drainage tube that is pressed. When operating in the negative-pressure environment or being pressed by an external force, the drainage tube 20 will be partially deformed, but the heteromorphic protrusions 201 of the drainage tube 20 help to keep the drainage effect and the negative-pressure environment 60 in the drainage tube 20. The heteromorphic protrusions 201 are designed to have a height ranging between 0.5 mm and 8 mm and a width ranging between 0.5 mm and 8 mm. This ensures that even when the tube wall of the drainage tube 20 and the heteromorphic protrusions 201 are pressed at the same time, a triangular space (gap) 203 can still be formed to keep the drainage effect and the negative-pressure environment 60.

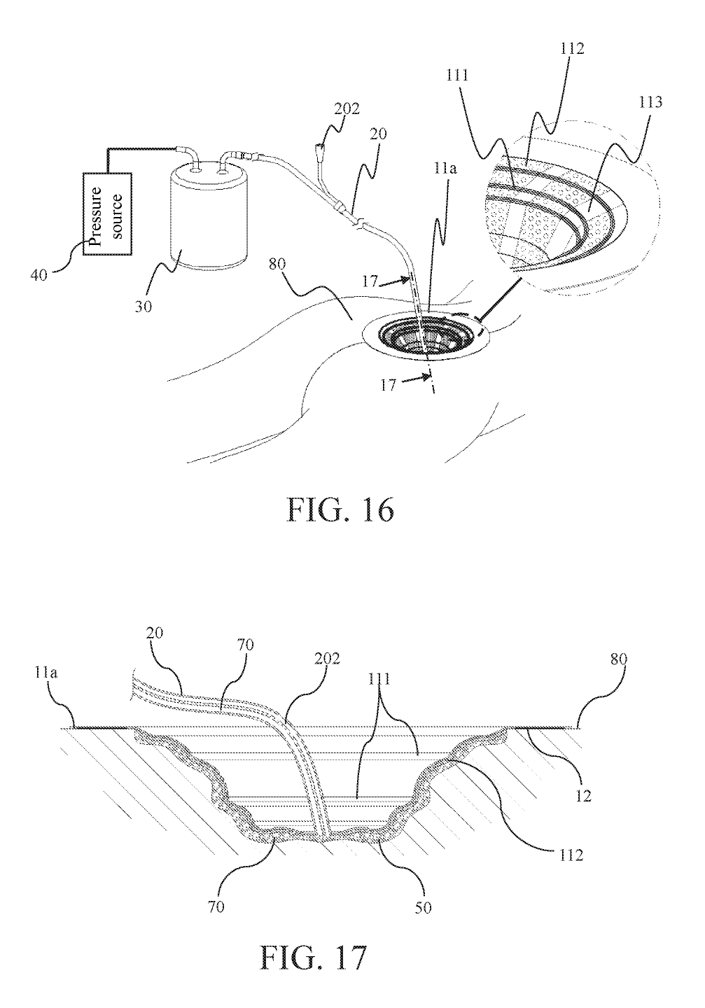

Please refer to FIG. 16 and FIG. 17. FIG. 16 is a schematic view of an irregularly-shaped coverage material of the closed-wound drainage device according to an embodiment. FIG. 17 is a schematic cross-sectional view of FIG. 16 taken along a cross-sectional line 17-17. The closed-wound drainage device 10 of the present invention is composed of a canister 30 and a pressure source 40. When being used for the treatment of a deeper wound, the closed-wound drainage device 10 is put on the wound tissue 50 in such a way that the foam 112 faces towards the wound tissue 50 and the adhesive layer 12 of the closed-wound drainage device 10 is attached on the normal skin 80 to provide a completely airtight effect for creating the negative-pressure environment 60. This is accomplished in two steps: firstly, the pressure source 40 is activated for pumping so that the irregularly-shaped coverage material 11a of the closed-wound drainage device 10 extends into the wound tissue 50 along with extension of the irregular loop 111, and then the infectious substances 70 generated in the wound tissue 50 can be pumped into the canister 30 via the foam gaps 112 of the foam 112 to facilitate the recovery of the wound. Secondly, an observation is made to the wound tissue 50 via the window 113 of the irregularly-shaped foamed coverage material 11, and if a large amount of infectious substances 70 are found in the wound tissue 50 or the wound is found to be inflamed, washing can be performed via the injection and washing port 202 of the drainage tube 20. The washing can be accomplished in two steps: firstly, the infectious substances 70 are drained through continuous pumping of the pressure source 40 in the injection and washing process; and secondly, the drug is injected into the injection and washing port 202 so that the drug diffuses to different sites of the wound tissue 50 via the foam 112 to provide an anti-inflammation and anti-bacterial effect.

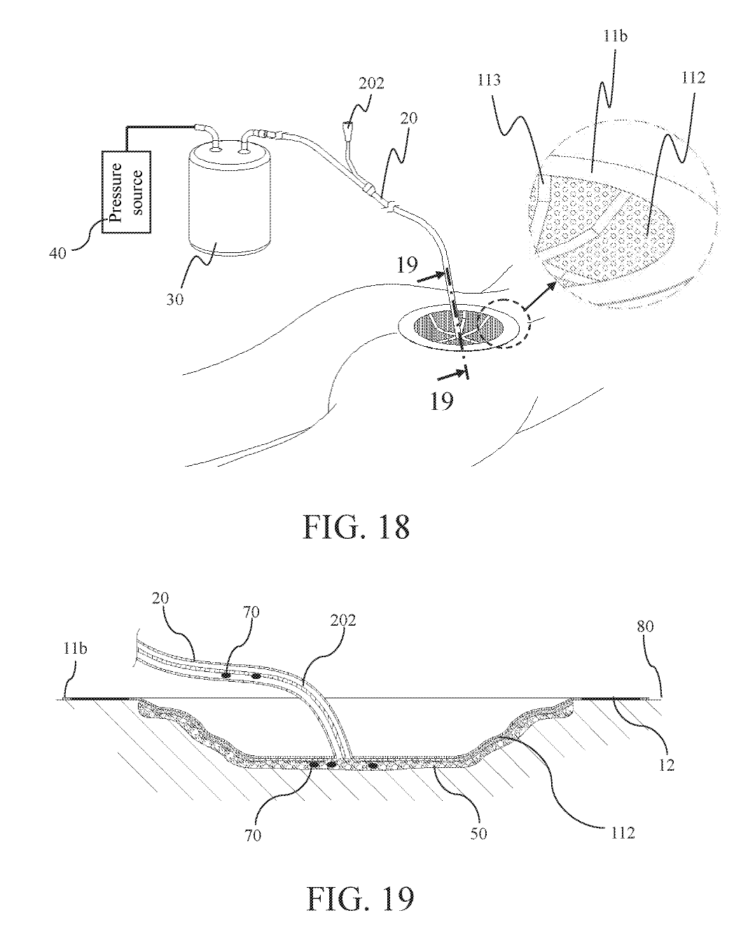

Please refer to FIG. 18 and FIG. 19. FIG. 18 is a schematic view of a flat coverage material of the closed-wound drainage device according to an embodiment, and FIG. 19 is a schematic cross-sectional view of FIG. 18 taken along a cross-sectional line 19-19. This implementation is used for treatment of a shallower wound in the following way: the closed-wound drainage device 10 is put on the wound tissue 50 in such a way that the foam 112 faces towards the wound tissue 50 and the adhesive layer 12 of the closed-wound drainage device 10 is attached on the normal skin 80 to provide a completely airtight effect in the negative-pressure environment 60. This is accomplished in two steps: first, the pressure source 40 is activated for pumping so that the flat coverage material 11b of the closed-wound drainage device 10 is attached to the wound tissue 50 by its extensibility, and then the infectious substances 70 generated in the wound tissue 50 can be pumped into the canister 30 via the foam gaps 112 of the foam 112 to facilitate recovery of the wound. Second, an observation is made to the wound tissue 50 via the window 113 of the flat foamed coverage material 11, and if a large amount of exudatives 70 and infectious substances 80 are found in the wound tissue 50 or the wound is found to be inflamed, washing can be performed via the injection and washing port 202 of the drainage tube 20, which is accomplished in two steps: first, the exudatives 70 and infectious substances 80 are drained through continuous pumping of the pressure source 40 in the injection and washing process; and second, the drug is injected into the injection and washing port 202 so that the drug diffuses to different sites of the wound tissue 50 via the foam 112 to provide an anti-inflammation and anti-bacterial effect.

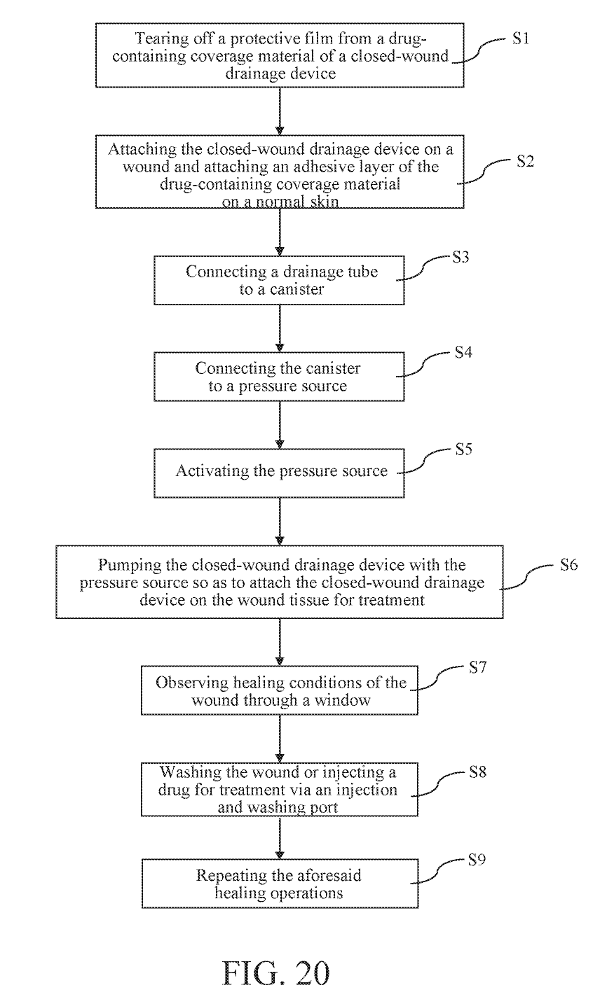

FIG. 20 is a schematic view of a flowchart diagram of a closed-wound drainage operation method. The operation method is as follows. As illustrated in step S1, a protective film is torn off from a drug-containing coverage material of a closed-wound drainage device. Then, in step S2, the closed-wound drainage device is attached on the wound and an adhesive layer of the drug-containing coverage material is attached on normal skin, where the drug-containing coverage material is an irregularly-shaped coverage material suitable for a wound of a deeper depth or a flat coverage material suitable for a wound of a shallower depth (see FIG. 3). Then, in step S3, a drainage tube is connected to a canister for collecting wasted liquid. In step S4, the canister is connected to a pressure source for creating a negative-pressure environment. Next, in step S5, the pressure source is activated to create a negative-pressure environment. In step S6, the closed-wound drainage device pumped by the pressure source is attached on the wound tissue in such a way that the foam is attached on the wound tissue along with the extension of the irregularly-shaped or flat coverage material, where the foam may drain the wasted liquid and also act as a drug carrier. Subsequently, in step S7, the recovery conditions of the wound is observed through a window (see FIG. 18). In step S8, the wound tissue is washed or a drug for treatment is injected via an injection and washing port so that when infectious substances are generated due to inflammation of the wound tissue, the wasted liquid of the infectious substances can be pumped into the canister via the pressure source by washing the infectious substances via the injection and washing port. Finally, in step S9, the aforesaid recovery operations are repeated.

What is described above is not intended to limit the scope of the present invention, and various alterations or modifications made by those skilled in the art without departing from the spirit and scope of the present invention shall all fall within the scope of the present invention. The scope of the present invention shall be defined by the claims as appended.

What is claimed is:

1. A closed-wound drainage device, comprising:
an irregularly-shaped or flat foam drug-containing coverage material, comprising at least one irregularly-shaped or flat flexible loop, at least one foam, a drug, and an adhesive layer, the at least one foam being a drainage channel or a drug carrier to drain, promote cell growth, and be antibacterial in order to accelerate recovery of a wound, and the adhesive layer being adhered to a skin to close the wound;
a drainage tube, comprising a plurality of heteromorphic protrusions to support a tube wall;
a canister, being adapted to collect exudatives and infectious substances generated by the wound tissue; and
a pressure source, providing a driving force for collecting the exudatives and infectious substances generated by the wound tissue;
wherein the at least one irregularly-shaped or flat flexible loop is adapted to be attached to wounds of various depths under the action of the pressure source; and
wherein the drainage tube connects with the canister and the pressure source, and when the pressure source is activated, the tube wall of the drainage tube provides a triangular space (gap) for smooth drainage.

2. The closed-wound drainage device according to claim 1, wherein the at least one irregularly-shaped or flat foam drug-containing coverage material has a thickness ranging between 0.05 mm and 2 mm.

3. The closed-wound drainage device according to claim 1, wherein the at least one irregularly-shaped or flat foam drug-containing coverage material has a hardness ranging between Shore A 20 and Shore A 80.

4. The closed-wound drainage device according to claim 1, wherein the at least one irregularly-shaped or flat foam drug-containing coverage material has an area ranging between 6 cm² and 400 cm².

5. The closed-wound drainage device according to claim 1, wherein the at least one irregularly-shaped or flat foam drug-containing coverage material has an extendable depth ranging between 1 mm and 150 mm.

6. The closed-wound drainage device according to claim 1, wherein the at least one foam of the at least one irregularly-shaped or flat foam drug-containing coverage material has a thickness ranging between 0.5 mm and 20 mm.

7. The closed-wound drainage device according to claim 1, wherein the at least one irregularly-shaped or flat foam drug-containing coverage material further comprises a window having a width ranging between 2 mm and 15 mm.

8. The closed-wound drainage device according to claim 1, wherein the heteromorphic protrusions of the drainage tube have a height ranging between 0.5 mm and 8 mm.

9. The closed-wound drainage device according to claim 1, wherein the heteromorphic protrusions of the drainage tube have a width ranging between 0.5 mm and 5 mm.

10. A closed-wound drainage device, comprising an irregularly-shaped or flat coverage material with a surface that has etched textures (chemically etched without dead spaces) for avoiding retention of infectious substances in dead spaces, and the etched textures have a width ranging between 0.01 mm and 5 mm and a depth ranging between 0.01 mm and 5 mm.

11. A closed-wound drainage operation method, comprising:
step S1: tearing off a protective film from a drug-containing coverage material of a closed-wound drainage device;
step S2: attaching the closed-wound drainage device on a wound tissue and attaching an adhesive layer of the drug-containing coverage material on a normal skin;
step S3: connecting a drainage tube to a canister for collecting wasted liquid;
step S4: connecting the canister to a pressure source for creating a negative-pressure environment;
step S5: activating the pressure source;
step S6: pumping the closed-wound drainage device with the pressure source so as to attach the closed-wound drainage device on the wound tissue in such a way that a foam of the drug-containing coverage material is attached on the wound tissue to drain the wasted liquid via a plurality of foam gaps comprised in the foam, wherein the foam is also able to act as a drug carrier;
step S7: observing recovery conditions of the wound through a window;
step S8: washing the wound tissue or injecting a drug for treatment via an injection and washing port; and
step S9: repeating the aforesaid recovery operations.

12. The closed-wound drainage operation method according to claim 11, wherein the drug-containing coverage material is an irregularly-shaped drug-containing coverage material suitable for a deeper wound or a flat drug-containing coverage material suitable for a shallower wound.
